(19) United States
(12) Patent Application Publication
(10) Pub. No.: US 2010/0121192 A1
(43) Pub. Date: May 13, 2010

(54) METHOD FOR IMAGING BLOOD VESSEL, SYSTEM FOR IMAGING BLOOD VESSEL AND PROGRAM FOR IMAGING BLOOD VESSEL

(75) Inventors: Fumio Nogata, Gifu (JP); Yasunari Yokota, Gifu (JP); Yoko Kawamura, Gifu (JP)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: GIFU UNIVERSITY, Gifu (JP)

(21) Appl. No.: 12/593,022

(22) PCT Filed: Mar. 21, 2008
(86) PCT No.: PCT/JP2008/055286

§ 371 Date: Nov. 11, 2009

(30) Foreign Application Priority Data
Mar. 28, 2007 (JP) 2007-084180

(51) Int. Cl. A61B 8/14

(52) U.S. Cl. 600/443

(57) ABSTRACT

The blood vessel imaging system includes a carriage for moving in a row direction an ultrasonic probe having a plurality of ultrasonic transducers arranged along a column direction and along the row direction. A computer repeats intermittent movement control for the carriage in shorter distance than a row pitch of the ultrasonic transducers along the row direction for each of a plurality of predetermined periods. Three-dimensional image processing or four-dimensional image processing is performed on the basis of a two-dimensional image and position information, the two-dimensional image being generated by making original image information, which is obtained by the ultrasonic transducers, two-dimensional in an ultrasonograph. The computer generates the images of a maximum diameter, a minimum diameter, and the difference between the maximum diameter and the minimum diameter for each measurement site in a carotid artery on the basis of the image processing result.

### TECHNICAL FIELD

The present invention relates to a method for imaging blood vessels, a system for imaging blood vessels, and a program for imaging blood vessels.

### BACKGROUND ART

Along with diversified lifestyles, progress of global aging, and diversified diet, arteriosclerosis has been progressing also among young people in recent years. Also, the number of patients receiving long-term therapy has been increasing due to vascular diseases such as myocardial infarction, cerebral infarction, and cerebral hemorrhage caused by arteriosclerosis. Mechanical integrity of vascular system, that is, flexibility of blood vessels (low rigidity and great breaking strain) is important in order to prevent the diseases.

A conventional method for measuring mechanical integrity of blood vessels in a noninvasive manner employs a pulse wave velocity measurement (PWM), blood vessel wall thickness measurement (intima-media thickness (IMT)) or the like as an arteriosclerosis examination. An elastic wave propagating through blood vessels is employed for the pulse wave velocity measurement because the propagation velocity of the elastic wave is proportional to an elastic coefficient of blood vessel. Further, the inner wall of blood vessel becomes gruel-like and if this state progresses, the inner wall expands. As a result, the blood vessel wall IMT increases and the inner diameter of the blood vessel decreases. The IMT (intima-media thickness) is examined at the first stage of the carotid echo examination.

Since the distance between two points used in the pulse wave velocity measurement is relatively great (for example, about ⅓ of the body height) when measuring these physical quantities, the measurement result takes an average value of measured lengths. In other words, it has been difficult to associate the progress status of locally occurred arteriosclerosis with the propagation velocity. For the blood vessel wall thickness measurement, special technique and knowledge are required for clear blood vessel imaging and wall thickness determination. Further, in recent years an acceleration pulse wave calculated from the 2nd order differential of a temporal change in blood stream of a fingertip or the like optically, but there is poor physical basis in which the arteriosclerosis can be measured from a change in blood stream near terminal blood vessels. Furthermore, a difference could be detected in PWM, IMT, acceleration pulse wave or the like when the arteriosclerosis was significantly advanced. Moreover, it is difficult to directly associate these measurement values with a rupture pressure of blood vessel hardened.

Patent Document 1 and Patent Document 2 do not disclose any technical feature for measuring a dynamic deformation behavior in a short time when the dynamic deformation behavior is measured for each measurement site in artery blood vessels.

Patent Document 1: Japanese Laid-Open Patent Publication No. 2005-185575

Patent Document 2: Japanese Laid-Open Patent Publication No. 2005-270351

### DISCLOSURE OF THE INVENTION

Changes occur in diameter of an artery along with a pulsatile flow caused by dilatation and contraction of the heart. FIG. 10(a) and FIG. 10(b) show a state in which the diameter of a carotid artery K changes from upstream toward downstream along with a pulsatile flow. However, when the inner wall of blood vessel changes in its nature and arteriosclerosis (increase in rigidity) occurs due to local increase in IMT for aging and adult diseases, the change in diameter along with the pulsatile flow causes less variation. Further, the arteriosclerosis may locally occur and a blood vessel in a three-layered structure is a material that exhibits a strong viscoelastic character in terms of material science. Thus, the rigidity defined in a physiological blood pressure variation region at rest (generally on the order of 70 to 140 mmHg) and a dynamic deformation behavior at a high pulsation speed (stress test) are measured and compared with each other, which gives an important indicator.

However, the technique for imaging a carotid artery blood vessel and for measuring deformability based on the conventional method has been unable the problem of measuring the above in a short time.

It is an objective of the present invention to provide a blood vessel imaging system, a blood vessel imaging program, and a blood vessel imaging method capable of remarkably reducing stress on a test subject when the rigidity defined in a physiological blood pressure variation region at rest and the dynamic deformation behavior at a high pulsation speed (stress test) are measured and compared with each other.

In order to solve the problems, according to one aspect of the present invention, there is provided a blood vessel imaging system comprising an ultrasonograph, a moving body, control means, position information input means, storage means, image processing means, and image generation means. The moving body integrally moves a group of ultrasonic transducers along a row direction, the ultrasonic transducers being arranged two-dimensionally, that is, along a column direction and along the row direction, and including a plurality of column-directional array ultrasonic transducers for creating a tomographic echo image. The control means repeats intermittent movement control for the moving body within a shorter distance than a row pitch of the ultrasonic transducers along the row direction for each of a plurality of predetermined periods. The position information input means receives position information on a stop position of the moving body. The storage means stores a two-dimensional image and the position information on the stop position in association with each other, the two-dimensional image being generated by making original image information, which is obtained by the ultrasonic transducers within the plurality of predetermined periods, two-dimensional in the ultrasonograph. The image processing means performs three-dimensional image processing based on the two-dimensional image and the position information, or performs temporal changing of a three-dimensional image, that is, four-dimensional image processing based on the two-dimensional image, the position information, and temporal information on the plurality of predetermined periods. The image generation means generates at least one image from among the images of a maximum diameter, a minimum diameter, and the difference between the maximum diameter and the minimum diameter for each measurement site in an artery blood vessel, that is, for each stop position on the basis of the image information subjected to the image processing in the image processing means.

According to this configuration, when the rigidity defined in a physiological blood pressure variation region at rest and the dynamic deformation behavior at a high pulsation speed (stress test) are measured and compared with each other, a stress on a test subject is remarkably reduced on the examination.

Preferably, the blood vessel imaging system further comprises heartbeat information input means for receiving heartbeat information. In this case, the control means employs a heartbeat period based on the heartbeat information as the predetermined period. According to this configuration, the heartbeat period can be used as the predetermined period to reliably acquire information on the maximum diameter and the minimum diameter of an artery blood vessel along with a heartbeat at a measurement site.

Preferably, the control means stops intermittent drive of the moving body when the total movement distance of the intermittently-driven moving body becomes equal to or greater than the row pitch of the ultrasonic transducers. With this configuration, since the intermittent drive of the moving body stops when the total movement distance of the intermittently-driven moving body becomes equal to or greater than the row pitch of the ultrasonic transducers, it is possible to efficiently measure the necessary sites in artery blood vessels. In other words, if the intermittent drive of the moving body does not stop when the total movement distance becomes equal to or greater than the row pitch of the ultrasonic transducers, unnecessary measurement is made, which may be inefficient.

Preferably, the blood vessel imaging system further comprises calculation means for calculating an elastic modulus of a measurement site on the basis of the difference between the maximum diameter and the minimum diameter for each measurement site in an artery blood vessel, that is, for each stop position and a previously input blood pressure at rest. According to this configuration, the elastic modulus of the measurement site is obtained on the basis of the difference between the maximum diameter and the minimum diameter for each measurement site in an artery blood vessel and the previously input blood pressure at rest, thereby obtaining deformability (hardness, arteriosclerosis) of the measurement site.

Preferably, the moving body comprises an ultrasonic probe having a plurality of ultrasonic transducers arranged along the row direction and the column direction. In this case, the ultrasonic probe comprises a concave face corresponding to a surface shape of the cervical region, and the plurality of ultrasonic transducers are arranged on the concave face. According to this configuration, the plurality of ultrasonic transducers are arranged along the row direction and along the column direction, respectively, on the concave face corresponding to the surface shape of the cervical region in the ultrasonic probe, thereby providing a blood vessel imaging system suitable for a carotid artery blood vessel of the cervical region.

According to another aspect of the present invention, there is provided a blood vessel imaging program for working a computer to function as control means, position information input means, storage means, image processing means and image generation means. The control means performs movement control for a moving body which integrally moves a group of ultrasonic transducers along a row direction, the ultrasonic transducers being arranged two-dimensionally, that is, along a column direction and the row direction, and including a plurality of column-directional array ultrasonic transducers for creating a tomographic echo image. The control means repeats intermittent movement control for the moving body within a shorter distance than a row pitch of the ultrasonic transducers along the row direction for each of a plurality of predetermined periods. The position information input means receives position information on a stop position of the moving body. The storage means stores a two-dimensional image and the position information on the stop position in association with each other, the two-dimensional image being generated by making original image information, which is obtained by the ultrasonic transducers within the plurality of predetermined periods, two-dimensional in the ultrasonograph. The image processing means performs three-dimensional image processing based on the two-dimensional image and the position information, or performs temporal changing of a three-dimensional image with time, that is, four-dimensional image processing based on the two-dimensional image, the position information, and temporal information on the plurality of predetermined periods. The image generation means generates at least one image from among the images of a maximum diameter, a minimum diameter and the difference between the maximum diameter and the minimum diameter for each measurement site in an artery blood vessel, that is, for each stop position on the basis of the image information subjected to the image processing in the image processing means.

With this configuration, when the rigidity defined in physiological blood pressure variation region at rest and the dynamic deformation behavior at a high pulsation speed (stress test) are measured and compared with each other, a stress on the test subject is remarkably reduced on the examination. Further, a function of displaying an arteriosclerosis distribution diagram of arteries can be added to an existing ultrasonograph, thereby enabling an artery examination in a short time and diagnosis assistance.

Preferably, the blood vessel imaging program works the computer to function as heartbeat information input means for receiving heartbeat information. In this case, a heartbeat period based on the heartbeat information is employed as the predetermined period. According to this configuration, the heartbeat period may be used as the predetermined period to reliably acquire information on the maximum diameter and the minimum diameter of an artery blood vessel along with a heartbeat at a measurement site.

Preferably, the control means stops intermittent drive of the moving body when a total movement distance of the intermittently-driven moving body becomes equal to or greater than the row pitch of the ultrasonic transducers. With this configuration, since the intermittent drive of the moving body stops when the total movement distance of the intermittently-driven moving body becomes equal to or greater than the row pitch of the ultrasonic transducers, it is possible to efficiently measure the necessary sites in artery blood vessels. In other words, if the intermittent drive of the moving body does not stop when the total movement distance becomes equal to or greater than the row pitch of the ultrasonic transducers, unnecessary measurement is made, which may be inefficient.

Preferably, the blood vessel imaging program works the computer to function as calculation means for calculating an elastic modulus of a measurement site on the basis of the difference between the maximum diameter and the minimum diameter for each measurement site in artery blood vessels, that is, for each stop position and a previously input blood pressure at rest. According to this configuration, the elastic modulus of the measurement site is obtained on the basis of the difference between the maximum diameter and the minimum diameter for each measurement site in artery blood vessels and the previously input blood pressure at rest, thereby obtaining deformability (hardness, arteriosclerosis) of the measurement site.

According to still another aspect of the present invention, there is provided a blood vessel imaging method. The blood vessel imaging method comprises a step of repeating intermittent movement control for the moving body which integrally moves a group of ultrasonic transducers along a row direction, the ultrasonic transducers being arranged two-dimensionally, that is, along a column direction and along the row direction, and including column-directional array ultrasonic transducers for creating a tomographic echo image, the intermittent movement control for the moving body being performed in a shorter distance than a row pitch of the ultrasonic transducers along the row direction for each of a plurality of predetermined periods. In this case, the blood vessel imaging method comprises a step of inputting position information on a stop position of the moving body and a step of storing a two-dimensional image and the position information on the stop position in association with each other, the two-dimensional image being generated by making original image information, which is obtained by the ultrasonic transducers in the plurality of predetermined periods, two-dimensional in the ultrasonograph. The blood vessel imaging method comprises a step of performing three-dimensional image processing based on the two-dimensional image and the position information, or performing temporal changing of a three-dimensional image with time, that is, four-dimensional image processing based on the two-dimensional image, the position information, and temporal information on the plurality of predetermined periods. Further, the blood vessel imaging method comprises a step of generating at least one image from among the images of a maximum diameter, a minimum diameter and the difference between the maximum diameter and the minimum diameter for each measurement site in an artery blood vessel, that is, for each stop position on the basis of the image information subjected to the image processing.

According to this configuration, when the rigidity defined in a physiological blood pressure variation region at rest and the dynamic deformation behavior at a high pulsation speed (stress test) are measured and compared with each other, a stress on the test subject is remarkably reduced on the examination. Further, a function of displaying an arteriosclerosis distribution diagram of arteries can be added to an existing ultrasonograph, thereby enabling an artery examination in a short time and diagnosis assistance.

### BRIEF DESCRIPTION OF THE DRAWINGS

FIG. 1 is a schematic diagram showing a configuration of a blood vessel imaging system according to a first embodiment;

FIG. 2 is a block diagram showing the blood vessel imaging system;

FIG. 3 is a schematic perspective view of a carriage and an ultrasonic probe;

FIG. 4 is a flowchart showing a processing of a blood vessel imaging program executed by a computer;

FIG. 5 is a diagram showing a three-dimensional image processing;

FIG. 6 is a graph showing changes in diameter of an artery blood vessel;

FIG. 7 is a schematic diagram showing a cross-sectional animation of an artery;

FIG. 8 is a schematic diagram showing a cross-sectional parallel image of an artery;

FIG. 9 is a diagram showing an arteriosclerosis analysis sheet;

FIGS. 10(a) and 10(b) are diagrams showing a state in which the diameter of a carotid artery is changing due to pulsation;

FIG. 11 is a schematic perspective view of a carriage and an ultrasonic probe according to a second embodiment; and

FIG. 12 is a schematic perspective view showing a carriage and an ultrasonic probe according to another example.

### BEST MODES FOR CARRYING OUT THE INVENTION

### First Embodiment

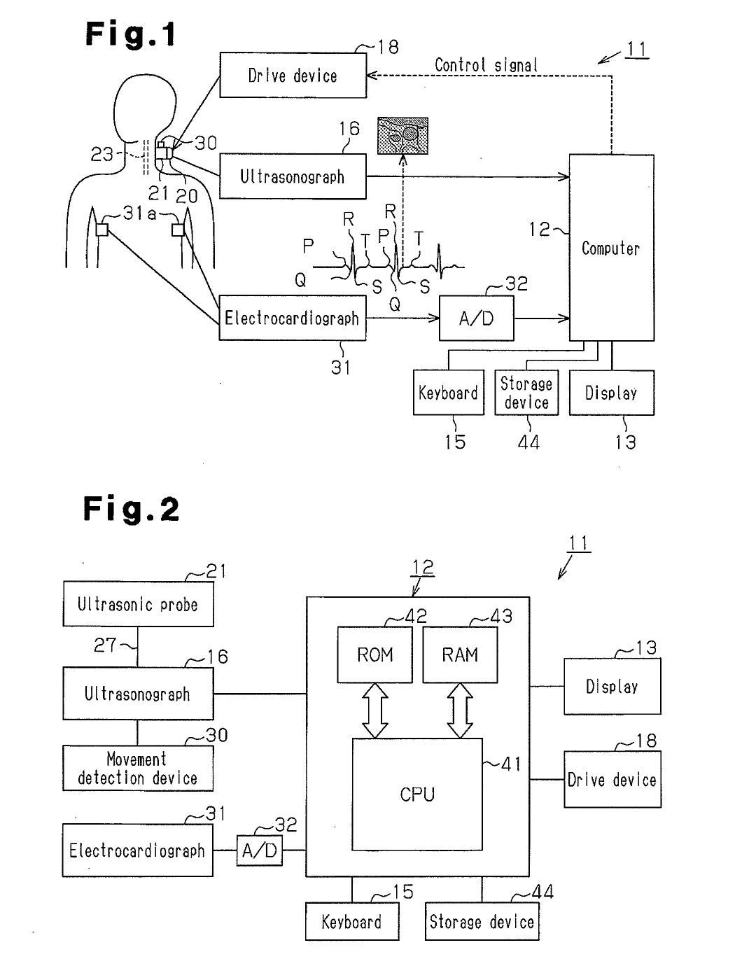

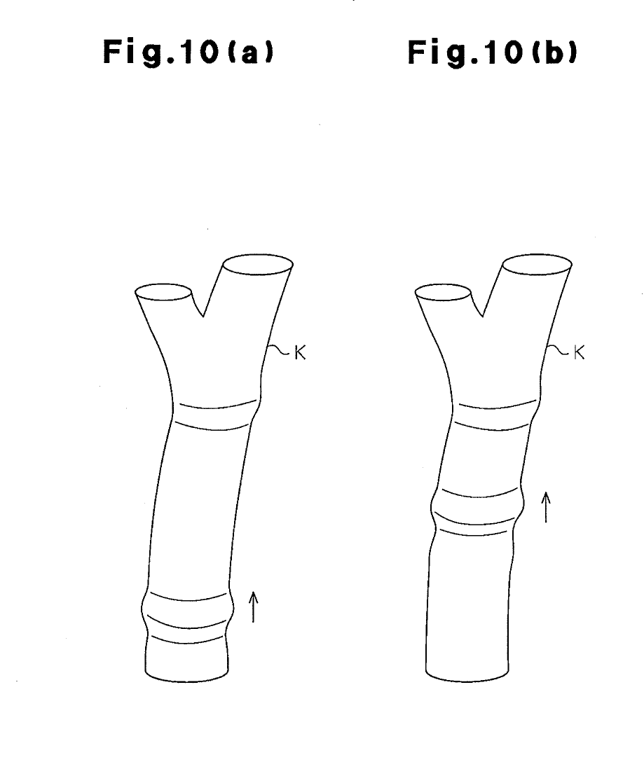

A blood vessel imaging system according to a first embodiment of the present invention will be described below with reference to FIGS. 1 to 10. As shown in FIGS. 1 and 2, a blood vessel imaging system 11 according to the present embodiment comprises a computer 12, a display 13, a keyboard 15, an ultrasonograph 16, and an electrocardiograph 31.

The blood vessel imaging system 11 analyzes the state of arteriosclerosis of a test subject on the basis of artery information of the test subject which is input from the keyboard 15 and the ultrasonograph 16 into the computer 12 or calculated by the computer 12, and outputs the analysis result of the arteriosclerosis to the display 13 and a printer (not shown) as output means. The artery information of the test subject includes carotid artery animation, blood pressure information, name information, ID (Identification) information, age information, and position information.

The carotid artery animation is information indicative of dilatation, contraction, and deformation of a carotid artery of the test subject, which is an image-processed three-dimensional image or four-dimensional image. In other words, the carotid artery animation is information indicative of a temporal change for at least one period in the dilatation, contraction, and deformation of the carotid artery which successively repeats the dilatation, contraction, and deformation. As shown in FIG. 1, the animation is obtained by a known pulse reflection method using the ultrasonograph 16.

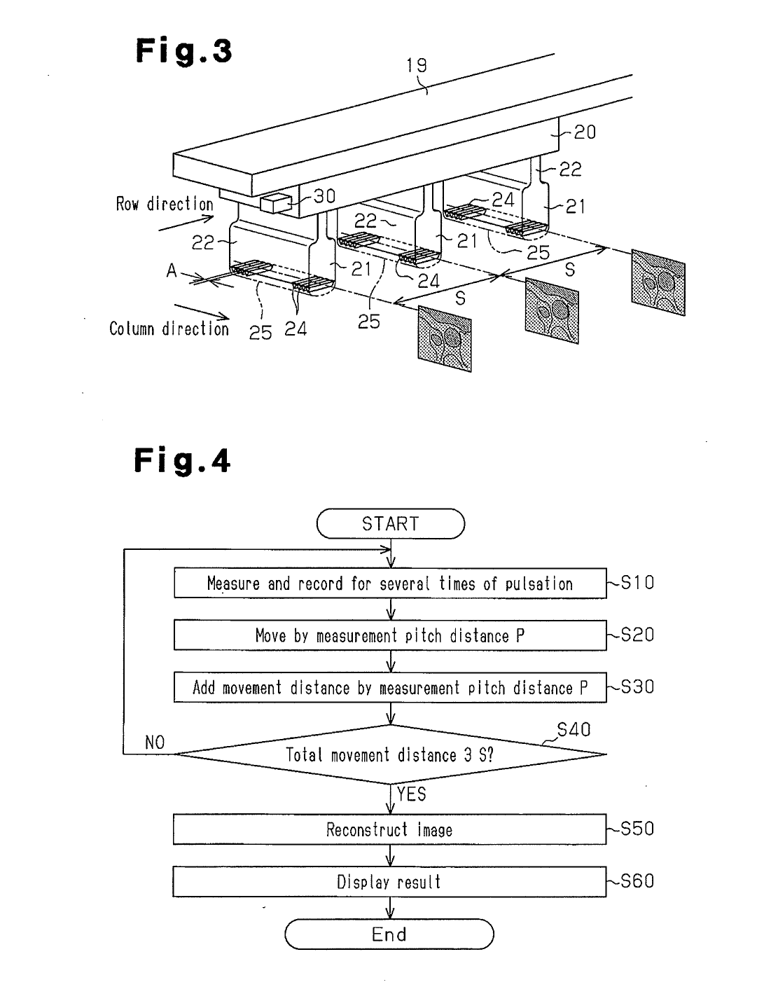

An ultrasonic probe 21 connected to the ultrasonograph 16 will be described. As shown in FIG. 3, in the present embodiment, a plurality of ultrasonic probes 21 are attached to the carriage 20 as a moving body to be supported on the carriage 20. The ultrasonic probe 21 comprises a probe body 22 constituted of a housing incorporating an ultrasonic transducer 24 for obtaining a tomographic image of a living body, and a cord 27 (see FIG. 2) extending from the probe body 22. The ultrasonic probe 21 is connected to the ultrasonograph 16 via the cord 27. In order to illustrate an arrangement state of the ultrasonic transducers 24 for purposes of illustration, FIG. 3 shows a state in which the ultrasonic transducers 24 are seen through from the ultrasonic probes 21.

An end face of the probe body 22 constitutes an ultrasonic emission face and the end face is provided with an acoustic lens 25. The ultrasonic transducer 24 of electronic scanning type made of piezoelectric device is arranged inside the probe body 22. Specifically, the probe body 22 is configured in a linear arrangement in which a plurality of ultrasonic transducers 24 are arranged linearly, that is, in lines at a pitch A. The ultrasonic transducers 24 are arranged along the column direction indicated by an arrow shown in FIG. 3. The ultrasonic scanning is performed along the column direction of the probe body 22. The adjacent ultrasonic probes 21 are arranged along the row direction orthogonal to the column direction to be separated at a pitch S, that is, at a row pitch. A value of the pitch S is on the order of 3 to 10 mm, for example. In this manner, each probe body 22 is provided with a column-directional array ultrasonic transducer made of a plurality of ultrasonic transducers for creating a tomographic echo image, and the plurality of ultrasonic probes 21 are arranged along the row direction.

The carriage 20 is supported on the rail 19 to be movable along the row direction, and is driven by a screw rod (not shown) rotated by a drive device 18. Since the carotid artery blood vessel is to be measured, the rail 19 according to the present embodiment is arranged in a direction in which the carotid artery blood vessel extends. The drive device 18 according to the present embodiment is configured in a servo motor, but may be configured in a step motor, for example.

The ultrasonograph 16 comprises a display unit and an ultrasonic source (neither shown). The ultrasonograph 16 transmits a pulse wave into a living body from a distal face of the ultrasonic probe 21 and receives a reflection wave (echo) from a carotid artery 23 (see FIG. 1) as original image information. The ultrasonograph 16 is configured such that the distal face of the ultrasonic probe 21 is formed in a rectangular shape, and a cross-sectional image of the carotid artery 23 is acquired as a tomographic echo image which is an echo animation acquired in the B/M mode. The display unit (not shown) of the ultrasonograph 16 displays thereon an animation as two-dimensional image when the carotid artery 23 of the test subject dilates, contracts or deforms. In other words, in the ultrasonograph 16, the display unit (not shown) displays thereon an artery's cross-sectional animation (two-dimensional image) as animation for the schematically-shown carotid artery 23, or the like. The artery's cross-sectional animation is an animation of the image indicative of the cross section in the diameter direction of the carotid artery 23.

The ultrasonograph 16 is connected to the computer 12, and the artery's cross-sectional animation (two-dimensional image, not shown) or the like acquired by the ultrasonograph 16 is input into the computer 12 and is stored in a storage device 44 as storage means to be associated with the ID information of the test subject.

A side of one ultrasonic probe 21 is provided with a movement detection device 30 as a position detection device. The movement detection device 30 comprises an image sensor (not shown) and a digital signal processor (DSP). The movement detection device 30 processes by the DSP an image of a test subject's body surface photographed by the image sensor to measure a movement distance from an arbitrary reference position on the test subject's body surface to the stop position of the ultrasonic probe 21. The movement distance is input into the computer 12 as position information. The position information is information indicative of the position of the site where the arteriosclerosis state is analyzed, that is, the position of the site where an animation on the carotid artery is acquired in the test object's body.

When the arteriosclerosis state of the carotid artery is analyzed, it is simplest if a bifurcation at which the common carotid artery bifurcates into the internal carotid artery and the external carotid artery is set as a reference point, for example. In the blood vessel imaging system 11, the reference point is previously input or stored in the computer 12. Then, a reference position of the body surface corresponding to the reference point is detected by the movement detection device 30, and the movement direction and the movement distance of the ultrasonic probe 21 relative to the reference position are detected by the movement detection device 30 so that the position information on the stop position is acquired. Further, the blood vessel imaging system 11 substantially successively acquires the position information on the stop position for one carotid artery 23 so that a detailed analysis result in which the position information and the arteriosclerosis analysis result for the carotid artery 23 are combined can be obtained. The information such as age information, name information, and ID information of the test subject is stored from the keyboard 15 into the storage device 44 of the computer 12 to be associated with each other.

The blood pressure information is configured with a maximum blood pressure (that is, maximum value) and a minimum blood pressure (that is, minimum value) at rest which are measured by a blood pressure measurement device (not shown). The blood pressure information is input into the computer 12 through the keyboard 15 or an electric signal relating to the blood pressure information from the blood pressure measurement device and is stored in the storage device 44 to be associated with the ID information of the test subject.

The heartbeat information is configured with an electrocardiogram measured by an electrocardiograph 31 whose electrodes 31a are attached to predetermined sites of the test subject's body as shown in FIG. 1 in the present embodiment. The heartbeat information is input into the computer 12 via an A/D 32 and is stored in the storage device 44 to be associated with the ID information of the test subject. The heartbeat information is not limited to the electrocardiogram and includes pulse wave, heart sound and the like in addition to the electrocardiogram. When the pulse wave is input, a sphygmograph is employed as the heartbeat information input means, and when the heart sound is input, a phonocardiograph is employed as the heartbeat information input means.

As shown in FIG. 1, a basic model of the electrocardiogram is composed of a spike called PQRST, where P indicates atrial excitation, QRS indicates ventricular excitation process, and T indicates ventricular excitation withdrawal process. A temporal relationship of the spike changes depending on the size of the heart and the heart rate. However, the temporal relationship of the spike in the case of an adult is substantially constant, and specifically PQ is on the order of 0.12 to 0.20 seconds, QRS is on the order of 0.05 to 0.08 seconds, and QT is on the order of 0.3 to 0.4 seconds.

A time between R and R is detected on the basis of the heartbeat information, for example, thereby obtaining a heartbeat period. The detection for obtaining the heartbeat period is not limited to the time between R and R, and for example, a time between P and P may be detected and a time between Q and Q may be detected. The heartbeat period corresponds to the predetermined period.

As shown in FIG. 2, the computer 12 comprises a CPU 41 (central processing unit), a ROM 42 and a RAM 43. The computer 12 executes a blood vessel imaging program stored in the ROM 42 to analyze an arteriosclerosis state based on the artery information, and performs an image processing on an animation as two-dimensional image when the carotid artery 23 of the test subject dilates, contacts or deforms to thereby generate a three-dimensional image or a four-dimensional image. The RAM 43 is a working memory when the program is being executed. The storage device 44 is configured with a hard disk or semiconductor storage device, for example, and is externally attached to the computer 12. Various information described above can be read from or written into the storage device 44.

The computer 12 having the storage device 44 corresponds to control means, position information input means, heartbeat information input means, storage means, image processing means, image generation means, and calculation means.

In the following, there will be described a processing of the blood vessel imaging program executed by the CPU 41 in the blood vessel imaging system 11 as configured above with reference to the flowchart of FIG. 4. The rail 19 is arranged in a direction in which the carotid artery blood vessel extends, each ultrasonic probe 21 is attached on the neck of the test subject, and the age information, the name information, the ID information and the blood pressure information of the test subject, for example, are input from the keyboard 15 to be stored in the storage device 44.

### (Step S10: Measurement and Recording for Several Times of Pulsation)

In step S10, the CPU 41 stores the image in the B/M mode from the ultrasonograph 16 and the position information from the movement detection device 30 into the storage device 44 for several times of pulsation, that is, for several heartbeats on the bases of the heartbeat information from the electrocardiograph 31. The number of heartbeats may be arbitrarily set and may be appropriately preset through the input from the keyboard 15. As a result, a tomographic image of an arbitrary position obtained by the ultrasonic probe 21 is used to record changes in blood vessel diameter for the several heartbeats along with pulsation (heartbeats) in the storage device 44. Therefore, the storage device 44 records therein a heartbeat period for the several heartbeats. The heartbeat period for the several heartbeats corresponds to temporal information on a plurality of predetermined periods. In this manner, the storage device 44 stores therein spatial information (three-dimensional information) of cross-sectional image and position information, and four-dimensional information such as temporal information on the plurality of predetermined periods.

A plurality of ultrasonic probes 21 are provided. Thus, with reference to the ultrasonic probe 21 to which the movement detection device 30 is attached, the cross-sectional image acquired by another adjacent ultrasonic probe 21 is associated with the position information to which the respective separation distances from the ultrasonic probe 21 as the reference are added, and is stored in the storage device 44.

### (Step S20: Movement of Measurement Pitch Distance P)

In step S20, the CPU 41 synchronizes with the heartbeat period on the basis of the heartbeat information from the electrocardiograph 31, outputs a control signal to the drive device 18, and drives the drive device 18 to move the carriage 20 by a measurement pitch distance P. Consequently, each ultrasonic probe 21 synchronizes with the heartbeat period to move on the rail 19 along the row direction by the measurement pitch distance P. The measurement pitch distance P is set at a minute distance (for example, 0.5 mm) shorter than the pitch S (for example, about 3 to 10 mm in the present embodiment), but the minute distance is not limited to 0.5 mm.

### (Step S30: Calculation of Total Movement Distance)

Next, the CPU 41 calculates a total movement distance nP. In the calculation of the total movement distance nP, in step S30, the measurement pitch distance P is added to the total movement distance nP previously calculated. An initial value of the total movement distance nP is set at 0, where n is the number of times of movement of the ultrasonic probe 21.

### (Step S40: Determination of Total Movement Distance)

Next, the CPU 41 determines whether or not the total movement distance nP is equal to or more than the pitch S (row pitch) in step S40. When the total movement distance nP is less than the pitch S (row pitch), the CPU 41 determines “NO” in step S40 and the processing returns to step S10. In other words, the processing returns to step S10 so that the processing in step S10 is subsequently repeated. When the total movement distance nP is equal to or more than the pitch S (row pitch), the CPU 41 determines “YES” in S40 and the processing proceeds to step S50.

Consequently, when the determination is “YES”, the four-dimensional information is stored in the storage device 44 for the entire region from the measurement site of the carotid artery blood vessel when the ultrasonic probe 21 is first positioned to the carotid artery blood vessel positioned in the pitch S.

### (Step S50: Reconstruction of Image)

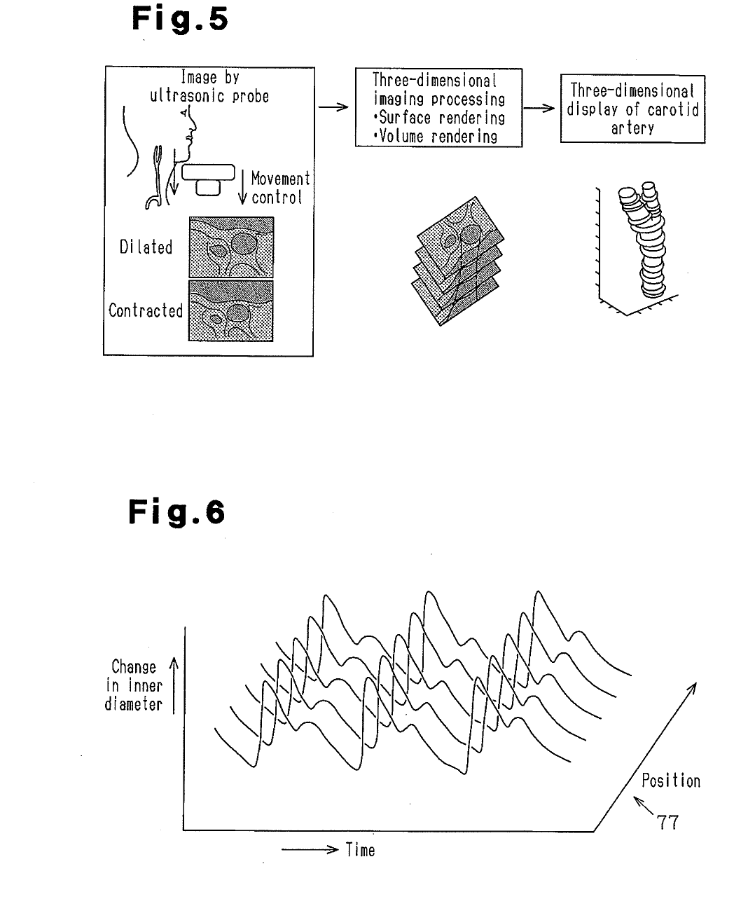

The CPU 41 performs three-dimensional image processing based on the two-dimensional image and the position information of the carotid artery blood vessel stored in the storage device 44, or performs four-dimensional image processing based on the two-dimensional image and the position information of the carotid artery blood vessel as well as the temporal information. In the case of the three-dimensional image processing, as shown in FIG. 5, the three-dimensional image processing is performed by surface rendering or volume rendering of the two-dimensional image (including the images at the time of dilatation and contraction) acquired by the ultrasonic probe 21 so that the carotid artery image is acquired. Further, if four-dimensioning is previously set, the CPU 41 performs the surface rendering or volume rendering of the two-dimensional image of the carotid artery blood vessel on the basis of the position information and the temporal information on predetermined time, and adds the temporal information to perform the four-dimensional image processing, thereby acquiring the carotid artery image. Moreover, the CPU 41 calculates an elastic modulus Eth.

### (Calculation of Elastic Modulus Eth)

The CPU 41 performs dimension analysis of the carotid artery for each site where each image is acquired on the basis of the carotid artery image.

### (Dimension Analysis of Carotid Artery)

The dimension analysis of the carotid artery will be described.

1. A Case where the Cross Section of the Carotid Artery is Circular

First, the case where the cross section of the carotid artery is circular will be described.

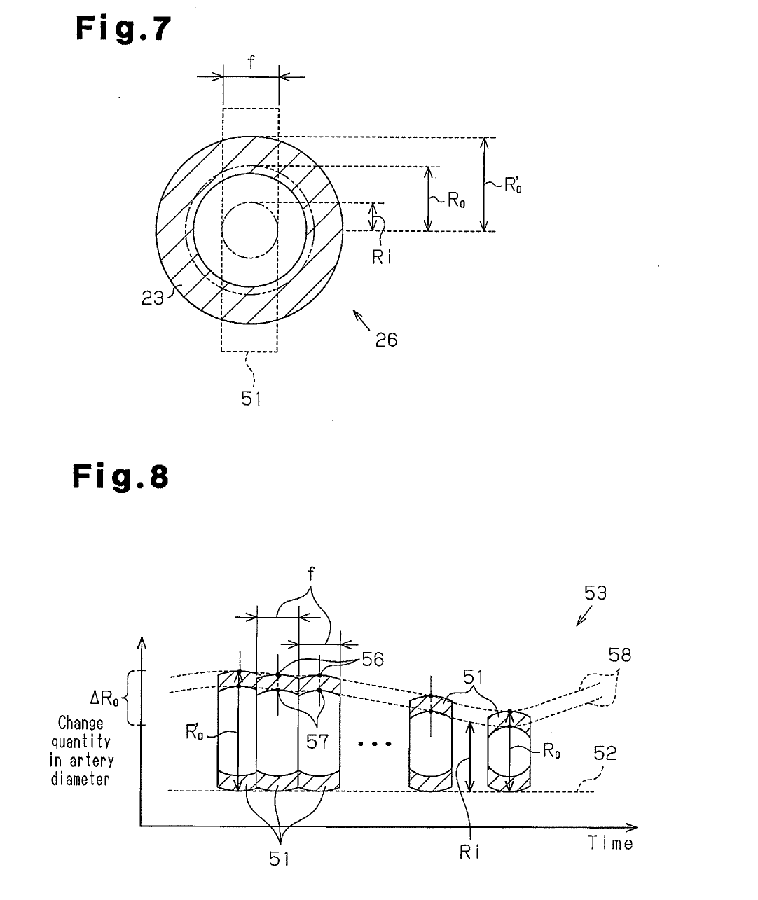

As shown in FIG. 7, the CPU 41 extracts an artery cross-sectional image 51 from an artery cross-sectional animation 26. The artery cross-sectional image 51 has a rectangular shape and a plurality of artery cross-sectional images 51 are extracted from the artery cross-sectional animation 26 for each constant time (for example, at an interval of about 0.05 seconds). Each artery cross-sectional image 51 is extracted such that the center of the carotid artery 23 is positioned on the center portion of the artery cross-sectional image 51, and at least the inner diameter and the outer diameter of the carotid artery 23 are set in a displayable size. The width f in a shorter direction in each artery cross-sectional image 51 is set to be equal among the respective artery cross-sectional images 51. The width f of the artery cross-sectional image 51 specifically corresponds to about several pixels (0.5 to 1 mm in the diameter direction of the carotid artery) on the display 13.

As shown in FIG. 8, the CPU 41 arranges the lower ends of the carotid arteries 23 in the respective artery cross-sectional images 51 along a reference line 52 to create an artery cross-sectional parallel image 53 as a parallel image. In the artery cross-sectional parallel image 53, each artery cross-sectional image 51 is arranged in parallel to be partially overlapped on another image in the order of extraction. The interval between the artery cross-sectional images 51 is set on the order of (width f)/2+several mm so that each artery cross-sectional image 51 is arranged such that the radius or diameter of the carotid artery 23 in the artery cross-sectional image 51 is displayed.

It is preferable that an image processing such as binarizing or contrast inversion is performed on the artery cross-sectional image 51. The carotid artery wall is made clear by the image processing so that the accuracy of the image analysis is improved when the blood vessel diameter is measured.

The CPU 41 creates a change curve indicative of a temporal change in carotid artery diameter from the artery cross-sectional parallel image 53. The CPU 41 connects midpoints 56 on the outer faces and midpoints 57 on the inner faces of the upper ends of the carotid arteries 23 in the respective artery cross-sectional images 51, respectively, in the artery cross-sectional parallel image 53 shown in FIG. 8 to create a lateral change curve as change curve indicated by broken lines in FIG. 8. Various numerical values for calculating the elastic modulus Eth are extracted from the lateral change curve 58. In other words, the CPU 41 extracts an outer radius R′₀ of the carotid artery 23 at the maximum dilatation, an outer radius R₀ and an inner radius Rᵢ of the carotid artery 23 at the maximum contraction as well as the difference ΔR₀ between the outer radius of the carotid artery 23 at the maximum dilatation and the outer radius of the carotid artery 23 at the maximum contraction from the lateral change curve 58 (see FIGS. 7 and 8).

2. A Case where the Cross Section of the Carotid Artery is Noncircular

The CPU 41 determines whether or not the cross section of the carotid artery 23 is noncircular, and when the cross section of the carotid artery 23 is noncircular, for example when the cross section thereof is oval, the CPU 41 calculates the radii (outer radius, inner radius) of the carotid artery 23 by the processing described later. In this case, when the distances from the center of gravity of the artery cross-sectional image 51 to a plurality of arbitrary points contained in the outer profile of the carotid artery 23 are equal to each other, the CPU 41 determines that the cross section is circular. A determination as to whether the cross section is circular or noncircular may be made by pattern matching.

The CPU 41 obtains an outer diameter area (area occupied by the outer profile of the carotid artery 23) of the carotid artery 23 from the cross-sectional image at the maximum dilatation and assumes a circle corresponding to the area. Then, the CPU 41 calculates a corresponding radius of the assumed circle and sets it to be the outer radius R′₀ of the carotid artery 23 at the maximum dilatation. Further, the CPU 41 obtains the outer diameter area (area occupied by the outer profile of the carotid artery 23) of the carotid artery 23 from the cross-sectional image at the maximum contraction and assumes a circle corresponding to the area. Then, the CPU 41 calculates a corresponding diameter of the assumed circle and sets it to be the outer radius R₀ of the carotid artery 23 at the maximum contraction. Further, the CPU 41 obtains an inner diameter area (area surrounded by the blood vessel inner wall of the carotid artery 23) of the carotid artery 23 from the cross-sectional image at the maximum contraction and assumes a circle corresponding to the area. Then, the CPU 41 calculates a corresponding diameter of the assumed circle and sets it to be the inner radius Rᵢ of the carotid artery 23 at the maximum contraction. Then, the CPU 41 extracts the difference ΔR₀ between the outer radius of the carotid artery 23 at the maximum dilatation and the outer radius of the carotid artery 23 at the maximum contraction.

### (Calculation of Elastic Modulus Eth)

The elastic modulus Eth is a parameter indicative of a mechanical property of the carotid artery, that is, rigidity, and is obtained from the temporal change in the carotid artery diameter and the blood pressure variation value of the test subject. Specifically, the CPU 41 uses the following expression (1) to calculate the elastic modulus Eth of each site of the carotid artery 23. The blood pressure variation value is calculated from the blood pressure information and indicates the difference between the maximum blood pressure (that is, maximum value) and the minimum blood pressure (that is, minimum value) of the test subject.

$$\begin{matrix}Expression 1 & \\ Elastic Modulus Eth = \frac{2 {R}_{0} ( Δ P ) {R}_{i}^{2}}{Δ {R}_{0} ( {R}_{0}^{2} - {R}_{i}^{2} )} & ( 1 )\end{matrix}$$

As described above, R₀ indicates the outer radius of the carotid artery 23 at the maximum contraction, Rᵢ indicates the inner radius of the carotid artery 23 at the maximum contraction, ΔP indicates the difference between the maximum blood pressure and the minimum blood pressure, and ΔR₀ indicates the difference between the outer radius of the carotid artery 23 at the maximum dilatation and the outer radius of the carotid artery 23 at the maximum contraction. The calculated elastic modulus Eth corresponds to the arteriosclerosis analysis result.

### (Step S60)

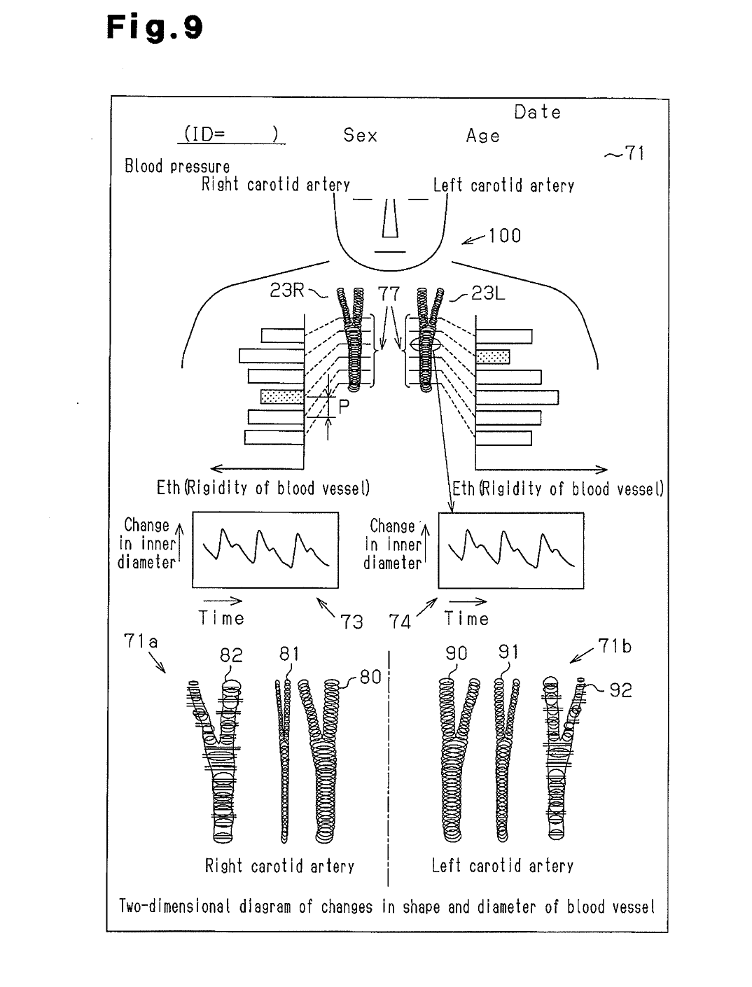

In step S60, the three-dimensional image or four-dimensional image subjected to the image processing in step S50 and the elastic modulus Eth are displayed on the display 13. In other words, the CPU 41 mutually combines and outputs the age information, the name information, the ID information as well as the elastic modulus Eth and the position information of the test subject and displays the elastic modulus Eth and the position information on the display 13. In the present embodiment, an arteriosclerosis analysis sheet 71 shown in FIG. 9 is displayed on the display 13. FIG. 9 exemplifies a case in which the right and left carotid arteries 23R and 23L are measured with 6 sites each. The carotid arteries 23R and 23L shown in FIG. 9 are subjected to the image processing to be illustrated as one example of the three-dimensional image.

The arteriosclerosis analysis sheet 71 indicates therein the upper body 100 of a person, the right and left carotid arteries 23R and 23L, position information 77, and the elastic modulus Eth indicated by a bar graph at each position where the carotid artery image is acquired as the arteriosclerosis distribution diagram.

In the present embodiment, the position information 77 is indicated at a position where the carotid artery image is acquired, that is, at a position of the measurement site (that is, the stop position of the carriage 20) with the bifurcation as a reference. The position information 77 is indicated so that the position of locally occurring arteriosclerosis can be easily identified. The arteriosclerosis analysis sheet 71 is printable by a printer (not shown) and the printed arteriosclerosis analysis sheet 71 can be provided to the test subject and doctors.

In the present embodiment, for the right and the left carotid arteries 23R and 23L, graphs 73 and 74 of the sites where the elastic modulus Eth is minimum are displayed in an easily viewable color different from the graphs of other sites so as to be paid more attention than other sites. The means for easy attraction is not limited to the display in a different color from other graphs. For example, the graph of a site where the elastic modulus Eth is minimum may be displayed in a blinking manner or may be displayed in a bar graph to be wider than other sites.

The arteriosclerosis analysis sheet 71 displays, for the right and left carotid arteries 23R and 23L, the graphs 73 and 74 for inner diameter displacement-time curve at the site where the elastic modulus Eth is minimum. FIG. 9 shows the graphs 73 and 74 for inner diameter displacement-time curve at the site where the elastic modulus Eth is minimum. However, as shown in FIG. 6, the position information 77 of each measurement site may be indicated to be lined with each other with respect to the reference position and the graphs for inner diameter displacement-time curve of the respective sites may be displayed.

The arteriosclerosis analysis sheet 71 is provided with the maximum and minimum display regions 71a and 71b of the diameter. The maximum and minimum display region 71a displays a maximum diameter image 80 of the inner diameter and a minimum diameter 81 of the inner diameter for the right carotid artery in the three-dimensional images and a two-dimensional diagram 82 of changes in the maximum diameter and the minimum diameter. The maximum and minimum display region 71b of the diameter displays a maximum diameter image 90 of the inner diameter and a minimum diameter 91 of the inner diameter for the left carotid artery in the three-dimensional images and a two-dimensional diagram 92 of changes in the maximum diameter and the minimum diameter. The two-dimensional diagram of the diameter change in the maximum diameter and the minimum diameter represents deformability of a blood vessel at each position. The two-dimensional diagram of the diameter change in the maximum diameter and the minimum diameter corresponds to an image of the difference between the maximum diameter and the minimum diameter.

In this manner, the arteriosclerosis analysis result of the carotid artery of the test subject is presented by means of the arteriosclerosis analysis sheet 71. When the arteriosclerosis analysis sheet 71 is printed by the printer (not shown), for the right and left carotid arteries 23R and 23L, the site where the elastic modulus Eth is minimum is printed in a easily viewable color different from the graphs of other sites in order to be paid more attention than other sites.

The present embodiment has the following advantages.

(1) The blood vessel imaging system 11 according to the present embodiment comprises the ultrasonograph 16 and the carriage 20 (moving body). The carriage 20 integrally moves a plurality of ultrasonic probes 21 along the row direction, which have a plurality of ultrasonic transducers 24 arranged along the column direction and are arranged along the row direction. The blood vessel imaging system 11 comprises the computer 12 (control means) for repeating intermittent movement control for the carriage 20 for each of a plurality of predetermined periods in a shorter distance than the pitch S (row pitch) of the ultrasonic transducer 24 along the row direction. The computer 12 comprises the storage device 44 to function as the position information input means, and the position information on the stop position of the carriage 20 is input into the computer 12.

The computer 12 functions as the storage means for storing the two-dimensional image and the position information on the stop position in association with each other, the two-dimensional image being generated by making the original image information, which is obtained by the ultrasonic transducer 24 in the plurality of predetermined periods, two-dimensional in the ultrasonograph 16. Further, the computer 12 functions as the image processing means for performing the three-dimensional image processing on the basis of the two-dimensional image and the position information or performing the four-dimensional image processing on the basis of the two-dimensional image, the position information and the temporal information on the plurality of predetermined periods. The computer 12 functions as the image generation means for generating the images of a maximum diameter, a minimum diameter and the difference between the maximum diameter and the minimum diameter for each measurement site in the carotid artery (artery blood vessel), that is, for each stop position on the basis of the image information subjected to the image processing.

As a result, the blood vessel imaging system 11 according to the present embodiment can make measurement of the elastic modulus Eth (rigidity) of the physiological blood pressure variation region (between the maximum blood pressure and the minimum blood pressure) at rest in a short time, thereby remarkably reducing a stress on the test subject.

Further, the blood vessel imaging system 11 according to the present embodiment can add to the existing ultrasonograph 16 the function of displaying an arteriosclerosis distribution diagram of arteries, that is, a distribution diagram in which the elastic modulus Eth and the position information 77 are combined as shown in FIG. 9, thereby enabling an artery examination in a short time and diagnosis assistance.

(2) In the present embodiment, the computer 12 of the blood vessel imaging system 11 receives the heartbeat information (electrocardiogram) as the heartbeat information input means, and the computer 12 functioning as the control means employs the heartbeat period based on the heartbeat information as a predetermined period. Consequently, the heartbeat period can be employed as a predetermined period to reliably acquire the information on the maximum diameter and the minimum diameter of the carotid artery (artery blood vessel) along with the heartbeat at the measurement site.

(3) In the present embodiment, the computer 12 functioning as the control means stops the intermittent drive of the carriage 20 when the total movement distance nP of the intermittently-driven carriage 20 becomes equal to or greater than the pitch S (row pitch) of the ultrasonic transducers 24.

As a result, since the intermittent drive of the carriage 20 stops when the total movement distance nP of the intermittently-driven carriage 20 becomes equal to or greater than the pitch S (row pitch) of the ultrasonic transducers 24, it is possible to efficiently measure the necessary sites of the carotid artery (artery blood vessel). In other words, if the intermittent drive of the carriage 20 does not stop when the total movement distance nP becomes equal to or greater than the pitch S of the ultrasonic transducers 24, unnecessary measurement is made, which may be inefficient.

(4) In the present embodiment, the computer 12 of the blood vessel imaging system 11 functions as the calculation means to calculate the elastic modulus Eth of the measurement site on the basis of the difference between the maximum diameter and the minimum diameter for each measurement site in the carotid artery (artery blood vessel), that is, for each stop position, and the previously input blood pressure at rest.

Consequently, the elastic modulus Eth of the measurement site can be obtained on the basis of the difference between the maximum diameter and the minimum diameter for each measurement site in the carotid artery (artery blood vessel) and the previously input blood pressure at rest, thereby obtaining deformability (hardness, arteriosclerosis) of the measurement site.

(5) The blood vessel imaging program according to the present embodiment works the computer 12 to function as the control means for performing movement control for the carriage 20 which integrally moves a group of ultrasonic transducers along the row direction, the control means repeating intermittent movement control for the carriage 20 within a shorter distance than the pitch S of the ultrasonic transducers 24 along the row direction for each of a plurality of predetermined periods. Further, the blood vessel imaging program works the computer 12 to function as the position information input means for receiving the position information on the stop position of the carriage 20. Moreover, the blood vessel imaging program works the computer 12 to function as the storage means for storing the two-dimensional image and the position information on the stop position in association with each other, the two-dimensional image being generated by making original image information, which is obtained by the ultrasonic transducer 24 within a plurality of predetermined periods, two-dimensional in the ultrasonograph 16.

Further, the blood vessel imaging program works the computer 12 to function as the image processing means for performing three-dimensional image processing on the basis of a two-dimensional image and position information or performing four-dimensional image processing on the basis of the two-dimensional image, the position information and the temporal information on a plurality of predetermined periods. The blood vessel imaging program works the computer 12 to function as the image generation means for generating the images of a maximum diameter, a minimum diameter and the difference between the maximum diameter and the minimum diameter for each measurement site in the artery blood vessel, that is, for each stop position on the basis of the image information subjected to the image processing.

Consequently, the blood vessel imaging program is capable of remarkably reducing a stress on the test subject when the rigidity of the physiological blood pressure variation region at rest is measured. Further, the blood vessel imaging program according to the present embodiment can add to the existing ultrasonograph 16 the function of displaying an arteriosclerosis distribution diagram of arteries, thereby enabling an artery examination in a short time and diagnosis assistance.

(6) The blood vessel imaging program according to the present embodiment works the computer 12 to function as the heartbeat information input means for receiving heartbeat information, and a heartbeat period based on the heartbeat information is employed as a predetermined period. As a result, the heartbeat period is employed as a predetermined period to reliably acquire the information on the maximum diameter and the minimum diameter of the carotid artery (artery blood vessel) along with the heartbeat at the measurement site.

(7) The blood vessel imaging program according to the present embodiment works the computer 12 to function as the control means for stopping the intermittent drive of the carriage 20 when the total movement distance nP of the intermittently-driven carriage 20 becomes equal to or greater than the pitch S of the ultrasonic transducers 24. As a result, since the intermittent drive of the carriage 20 stops when the total movement distance nP of the intermittently-driven carriage 20 becomes equal to or greater than the pitch S of the ultrasonic transducer 24, measurement of the necessary sites of the carotid artery (artery blood vessel) can be efficiently made. In other words, if the intermittent drive of the carriage 20 does not stop when the total movement distance nP becomes equal to or greater than the pitch S of the ultrasonic transducer 24, unnecessary measurement is made, which may be inefficient.

(8) The blood vessel imaging program according to the present embodiment works the computer 12 to function as the calculation means for calculating the elastic modulus Eth of the measurement site on the basis of the difference between the maximum diameter and the minimum diameter for each measurement site in the artery blood vessel, that is, for each stop position, and the previously input blood pressure at rest. As a result, the elastic modulus Eth of the measurement site can be obtained on the basis of the difference between the maximum diameter and the minimum diameter for each measurement site in the carotid artery (artery blood vessel) and the previously input blood pressure at rest, thereby obtaining deformability (hardness, arteriosclerosis) of the measurement site.

(9) The blood vessel imaging method according to the present embodiment comprises the step of repeating intermittent movement control for the carriage 20 which integrally moves a group of ultrasonic transducers along the row direction, the ultrasonic transducers including a plurality of ultrasonic transducers 24 arranged two-dimensionally, that is, along the column direction and the row direction, respectively, the intermittent movement control for the carriage 20 being performed within a shorter distance than the pitch S of the ultrasonic transducers 24 along the row direction for each of a plurality of predetermined periods. Further, the blood vessel imaging method comprises the step of receiving position information on a stop position of the carriage 20, and the step of storing the two-dimensional image and the position information on the stop position in association with each other, the two-dimensional image being generated by making original image information, which is obtained by the ultrasonic transducer 24 within a plurality of predetermined periods, two-dimensional in the ultrasonograph 16.

Moreover, the blood vessel imaging method comprises the step of performing three-dimensional image processing on the basis of a two-dimensional image and position information or performing four-dimensional image processing on the basis of the two-dimensional image, the position information and the temporal information on a plurality of predetermined periods. Additionally, the blood vessel imaging method comprises the step of generating the images of a maximum diameter, a minimum diameter and the difference between the maximum diameter and the minimum diameter for each measurement site in the artery blood vessel, that is, for each stop position on the basis of the image information subjected to the image processing.

Consequently, there can be provided the blood vessel imaging method capable of remarkably reducing a stress on the test subject when measuring the rigidity of the physiological blood pressure variation region at rest. Further, the function of displaying the arteriosclerosis distribution diagram of the arteries can be added to the existing ultrasonograph, thereby enabling an artery examination in a short time and diagnosis assistance.

### Second Embodiment

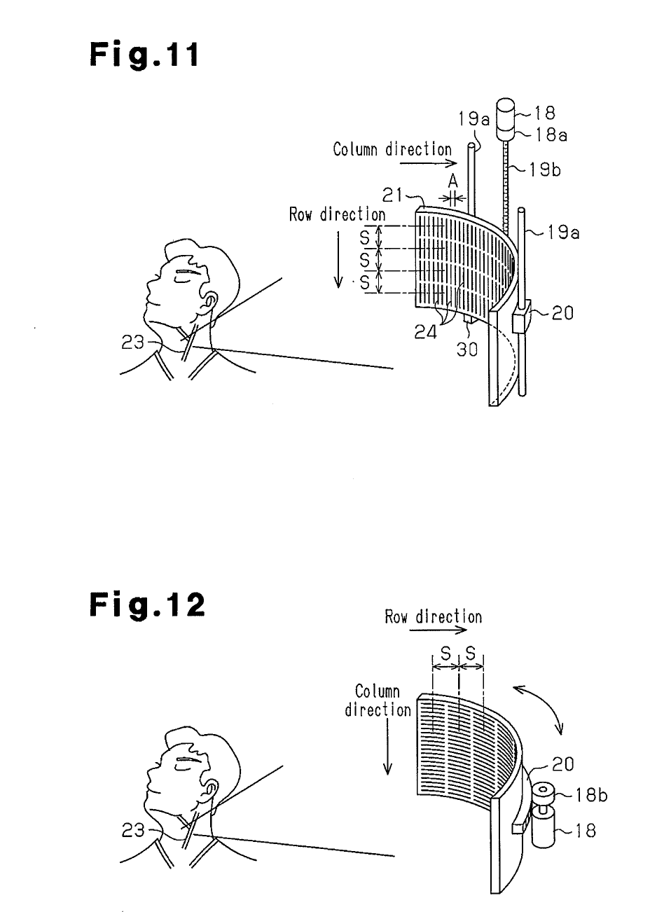

In the following, a blood vessel imaging system according to a second embodiment of the present invention will be described with reference to FIG. 11. The same numerals are denoted to the same constituents as those in the first embodiment to omit the description thereof, and different configurations from the first embodiment will be mainly explained in the following description.

Specifically, in the second embodiment, the configurations of the ultrasonic probe 21 and the carriage 20 are different from those in the first embodiment and other configurations are the same as those in the first embodiment. Thus, the configurations of the ultrasonic probe 21 and the carriage 20 will be described.

As shown in FIG. 11, the ultrasonic probe 21 is formed into a concave cross-sectional shape extending along the surface shape of the cervical region. In the ultrasonic probe 21, a plurality of groups of ultrasonic transducers 24, in which a plurality of ultrasonic transducers 24 are arranged in lines at a pitch A to be configured in a linear arrangement, are arranged at a pitch S (row pitch) in the concave surface opposed to the surface of the cervical region. In other words, a plurality of ultrasonic transducers 24 are arranged along the column direction and along the row direction, respectively. In the following description, the concave face of the ultrasonic probe 21 opposed to the surface of the cervical region is referred to as inner face. The row direction is a direction in which the carotid artery 23 extends and the column direction is a direction extending along the curvature direction of the inner face of the ultrasonic probe 21.

The ultrasonic probe 21 is provided with the carriage 20 at an outer face opposed to the inner face. A pair of rod-shaped guide rails 19a fixed on a fixing portion (not shown), which extends in parallel to the direction in which the carotid artery 23 extends, is slidably passed through the carriage 20. A screw rod 19b arranged in parallel to the guide rail 19a passes through a nut portion (not shown) provided on the carriage 20 to be meshed with the nut portion. The screw rod 19b is connected with the drive device 18 via a decelerator 18a. The ultrasonic probe 21 is provided with the movement detection device 30.

The rotation of the drive device 18 is controlled by the computer 12 so that the ultrasonic probe 21 is subjected to the intermittent movement control along the row direction at the pitch S.

The second embodiment has the following advantage in addition to the above advantages of the first embodiment.

(10) In the second embodiment, the carriage 20 comprises the ultrasonic probe 21 having a plurality of ultrasonic transducers 24 arranged along the column direction and along the row direction, respectively. The ultrasonic probe 21 comprises a concave face corresponding to the surface shape of the cervical region, and the plurality of ultrasonic transducers 24 are arranged on the face along the column direction and along the row direction, respectively. As a result, the blood vessel imaging system suitable for the carotid artery blood vessel of the cervical region can be provided.

The respective embodiments described above may be changed as follows.

The configuration of the ultrasonic probe 21 according to the second embodiment may be changed as shown in FIG. 12. In the configuration shown in FIG. 12, the shape of the ultrasonic probe 21 is set similarly as in the second embodiment, and a plurality of groups of ultrasonic transducers 24 which are arranged in lines at pitch A to be configured in a linear arrangement are arranged at the inner face at the pitch S (row pitch). Specifically, a plurality of ultrasonic transducers 24 are arranged along the column direction and along the row direction. The column direction is a direction in which the carotid artery 23 extends and the row direction is a direction extending in the curvature direction of the inner face of the ultrasonic probe 21.

A rack is formed on the outer face of the carriage 20 and a pinion 18b fixed on an output shaft of the drive device 18 is meshed with the rack. Then, the rotation of the drive device 18 is controlled by the computer 12 so that the ultrasonic probe 21 is subjected to the intermittent movement control in the row direction at the pitch S. Also with the configuration, similar operational effects as in the second embodiment can be obtained.

In the respective embodiments, for the measurement of the rigidity of the physiological blood pressure variation region at rest, the elastic modulus Eth is measured (calculated) using the maximum blood pressure (that is, maximum value) and the minimum blood pressure (that is, minimum value) at rest and is displayed. Instead, the dynamic deformation behavior in a state where a stress is imposed on the test subject, that is, at a high pulsation speed (stress test) may be measured and compared with the measurement result of the dynamic deformation behavior at rest with less stress, and the result may be displayed on the display 13. In this case, the computer 12 receives the blood pressure information in a stress-added state similarly as in the above embodiments. According to this configuration, when the dynamic deformation behavior at the high pulsation speed (stress test) is measured to be compared, a stress on the test subject is remarkably reduced.

The respective embodiments described above employ the heartbeat period as predetermined time but the predetermined time is not limited to the heartbeat period and a period longer than the heartbeat period may be employed.

The arteriosclerosis analysis result may be output from either one of the display 13 or the printer (not shown).

The present embodiments employ bar graphs, but line plots may be employed.

In the respective embodiments, the externally-attached storage device 44 is provided so that the computer 12 functions as the storage means. However, a storage device incorporated in the computer 12 may be employed. Further, a hard disk or memory made of semiconductor device may be employed as the storage device.

The computer 12 according to the respective embodiments generates the images of a maximum diameter, a minimum diameter and the difference between the maximum diameter and the minimum diameter for each measurement site in the carotid artery (artery blood vessel), that is, for each stop position on the basis of the image information subjected to the image processing. Alternatively, there may be generated only the image of the maximum diameter of the inner diameter for each stop position of the measurement site in the carotid artery (artery blood vessel), only the image of the minimum diameter thereof, or only the image of the difference between the maximum diameter and the minimum diameter. Further, the images of the maximum diameter and the minimum diameter of the inner diameter for each stop position of the measurement site in the carotid artery (artery blood vessel) may be generated, the image of the maximum diameter of the inner diameter and the image of the difference between the maximum diameter and the minimum diameter may be generated, or the image of the minimum diameter of the inner diameter and the image of the difference between the maximum diameter and the minimum diameter may be generated.

It has been intended the carotid artery in the respective embodiments described above, however any arteries imaging by ultrasonograph can also be applied.

1. A blood vessel imaging system comprising:
an ultrasonograph;
a moving body which integrally moves a group of ultrasonic transducers along a row direction, the ultrasonic transducers being arranged two-dimensionally, that is, along a column direction and along the row direction, and including a plurality of column-directional array ultrasonic transducers for creating a tomographic echo image;
control means for repeating intermittent movement control for the moving body in a shorter distance than a row pitch of the ultrasonic transducers along the row direction for each of a plurality of predetermined periods;
position information input means for receiving position information on a stop position of the moving body;
storage means for storing a two-dimensional image and the position information on the stop position in association with each other, the two-dimensional image being generated by making original image information, which is obtained by the ultrasonic transducers within the plurality of predetermined periods, two-dimensional in the ultrasonograph;
image processing means for performing three-dimensional image processing based on the two-dimensional image and the position information or performing temporal changing of a three-dimensional image with time, that is, four-dimensional image processing based on the two-dimensional image, the position information, and temporal information on the plurality of predetermined periods; and
image generation means for generating at least one image from among the images of a maximum diameter, a minimum diameter, and the difference between the maximum diameter and the minimum diameter for each measurement site in an artery blood vessel, that is, for each stop position on the basis of the image information subjected to the image processing in the image processing means.

2. The blood vessel imaging system according to claim 1, further comprising heartbeat information input means for receiving heartbeat information,
wherein the control means employs a heartbeat period based on the heartbeat information as the predetermined period.

3. The blood vessel imaging system according to claim 1, wherein the control means stops intermittent drive of the moving body when a total movement distance of the intermittently-driven moving body becomes equal to or greater than the row pitch of the ultrasonic transducers.

4. The blood vessel imaging system according to claim 1, further comprising calculation means for calculating an elastic modulus of a measurement site on the basis of the difference between the maximum diameter and the minimum diameter for each measurement site in the artery blood vessel, that is, for each stop position, and previously input blood pressures at rest.

5. The blood vessel imaging system according to claim 1, wherein the moving body comprises an ultrasonic probe having a plurality of ultrasonic transducers arranged along the column direction and along the row direction, and
wherein the ultrasonic probe comprises a concave face corresponding to a surface shape of the cervical region, and the plurality of ultrasonic transducers are arranged on the concave face.

6. A blood vessel imaging program for working a computer to function as:
control means for performing movement control for a moving body which integrally moves a group of ultrasonic transducers along a row direction, the ultrasonic transducers being arranged two-dimensionally, that is, along a column direction and along the row direction, and including a plurality of column-directional array ultrasonic transducers for creating tomographic echo image, the control means repeating intermittent movement control for the moving body in a shorter distance than a row pitch of the ultrasonic transducers along the row direction for each of a plurality of predetermined periods;
position information input means for receiving position information on a stop position of the moving body;
storage means for storing a two-dimensional image and the position information on the stop position in association with each other, the two-dimensional image being generated by making original image information, which is obtained by the ultrasonic transducers within the plurality of predetermined periods, two-dimensional in the ultrasonograph;
image processing means for performing three-dimensional image processing based on the two-dimensional image and the position information or performing temporal changing of a three-dimensional image with time, that is, four-dimensional image processing based on the two-dimensional image, the position information, and temporal information on the plurality of predetermined periods; and
image generation means for generating at least one image from among the images of a maximum diameter, a minimum diameter, and the difference between the maximum diameter and the minimum diameter for each measurement site in an artery blood vessel, that is, for each stop position on the basis of the image information subjected to the image processing in the image processing means.

7. The blood vessel imaging program according to claim 6, for working the computer to function as heartbeat information input means for receiving heartbeat information,
wherein a heartbeat period based on the heartbeat information is employed as the predetermined period.

8. The blood vessel imaging program according to claim 6, wherein the control means stops intermittent drive of the moving body when a total movement distance of the intermittently-driven moving body becomes equal to or greater than the row pitch of the ultrasonic transducers.

9. The blood vessel imaging program according to claim 6, for working the computer to function as calculation means for calculating an elastic modulus of a measurement site on the basis of the difference between the maximum diameter and the minimum diameter for each measurement site in the artery blood vessel, that is, for each stop position, and a previously input blood pressure at rest.

10. A blood vessel imaging method comprising the steps of:
repeating intermittent movement control for a moving body which integrally moves a group of ultrasonic transducers along a row direction, the ultrasonic transducers being arranged two-dimensionally, that is, along a column direction and along the row direction, and including column-directional array ultrasonic transducers for creating a tomographic echo image, the intermittent movement control for the moving body being performed in a shorter distance than a row pitch of the ultrasonic transducers along the row direction for each of a plurality of predetermined periods;
inputting position information on a stop position of the moving body;
storing a two-dimensional image and the position information on the stop position in association with each other, the two-dimensional image being generated by making original image information, which is obtained by the ultrasonic transducers within the plurality of predetermined periods, two-dimensional in the ultrasonograph;
performing three-dimensional image processing based on the two-dimensional image and the position information or performing temporal changing of a three-dimensional image with time, that is, four-dimensional image processing based on the two-dimensional image, the position information, and temporal information on the plurality of predetermined periods; and
generating at least one image from among the images of a maximum diameter, a minimum diameter, and the difference between the maximum diameter and the minimum diameter for each measurement site in an artery blood vessel, that is, for each stop position on the basis of the image information subjected to the image processing.
